(19) United States
(12) Patent Application Publication
(10) Pub. No.: US 2009/0175762 A1
(43) Pub. Date: Jul. 9, 2009

(54) OXYGENATOR UNIT AND OXYGENATOR APPARATUS

(75) Inventors: Mitsuaki Ogihara, Shizuoka (JP); Norikazu Ishida, Shizuoka (JP); Kazuhiko Takeuchi, Shizuoka (JP)

Correspondence Address: BUCHANAN, INGERSOLL & ROONEY PC, POST OFFICE BOX 1404, ALEXANDRIA, VA, 22313-1404, US

(73) Assignee: Terumo Kabushiki Kaisha, Shibuya-ku (JP)

(21) Appl. No.: 12/349,252

(22) Filed: Jan. 6, 2009

(30) Foreign Application Priority Data
Jan. 8, 2008 (JP) 2008-001358

(51) Int. Cl. A61M 1/32

(52) U.S. Cl. 422/45; 422/46

(57) ABSTRACT

An oxygenator unit includes a venous reservoir, a centrifugal pump, an oxygenator, a connection part for connecting the reservoir and the pump, a connection part for connecting the pump and the oxygenator, and a unit frame. The unit frame includes a reservoir receiving part, a pump receiving part provided at the position under a blood outlet of the venous reservoir disposed at the reservoir receiving part, and an oxygenator receiving part provided at the position in the vicinity of the blood outlet of the centrifugal pump disposed on the pump receiving part. The reservoir is disposed at the reservoir receiving part, the pump is disposed at the pump receiving part; and the oxygenator is disposed at the oxygenator receiving part, and they are held by the unit frame, resulting in a unit.

### TECHNOLOGICAL FIELD

The present invention generally relates to a device for oxygenating blood. More specifically, the invention pertains to an oxygenator unit and an oxygenator apparatus for use in heart surgery.

### BACKGROUND DISCUSSION

An oxygenator apparatus is used during heart surgery and is typically positioned in an extracorporeal circuit. The apparatus performs oxygenation of the blood extracted from a patient. The oxygenator apparatus comes in various types based on, for example, the type of oxygenator, the type of pump, and the difference in mounting position of the pump. The oxygenator apparatus generally includes a reservoir (blood reservoir), an oxygenator, a heat exchanger, a pump, and a plurality of tubes establishing connection between the various components.

to render the oxygenator apparatus usable, an arrangement operation of the reservoir (blood reservoir), the oxygenator, the heat exchanger and the pump is required as is a connection operation of the components. During heart surgery requiring an oxygenator apparatus, urgency may be demanded. Further, even when urgency is not demanded, it is desirable that the preparation operation be quickly completed.

Japanese Patent No. 3738211 (Patent Document 1) proposes an extracorporeal circuit system. The extracorporeal circuit system is a packed unit of an extracorporeal circuit system including an extracorporeal circuit system packed in one package. The extracorporeal circuit system has a venous reservoir, an oxygenator, an arterial filter, a reservoir holder for holding the venous reservoir, an oxygenator holder for holding the oxygenator, and a filter holder for holding the arterial filter. The venous reservoir and the oxygenator are connected with a first tube. The oxygenator and the arterial filter are connected to a second tube. A circulation pump for supplying the blood in the venous reservoir to the oxygenator is connected to the first tube. A blood extraction tube connection part capable of connecting a blood extraction tube to the venous reservoir is provided. A blood supply tube connection part capable of connecting a blood supply tube to the arterial filter is provided. The filter holder is rotatably connected to the side part of the reservoir holder. The filter holder is rotated such as to be along the side of the reservoir during storage, and such as to extend to the side part of the reservoir holder during use.

Conventionally, the arrangement of a venous reservoir, an oxygenator, a pump and the like in an extracorporeal circuit has not been unified or standardized according to the arrangement of devices in an operating room, the habit of the operator, and the like. Thus, the arrangement of the components may often vary depending upon the hospital and the operator. However, some uses of the pump oxygenator apparatus require urgency as described above.

### SUMMARY

An oxygenator unit disclosed here includes a venous reservoir, a pump and heat exchange unit-equipped oxygenator and the centrifugal pump are connected by the first connection part, and the centrifugal pump and the heat exchange unit-equipped oxygenator are connected by the second connection part. Further, the unit includes the unit frame. In addition, the venous reservoir is disposed at the reservoir disposition part and is held by the unit frame; the centrifugal pump is disposed at the pump disposition part and is held by the unit frame; and the heat exchange function-equipped oxygenator is disposed at the oxygenator disposition part and is held by the unit frame, thereby resulting in a unit.

With this disclosed construction, the positions of the venous reservoir, the oxygenator, and the pump cannot be changed arbitrarily. However, these pieces are set in a unit by the unit frame, and are arranged in a usable state. Accordingly, the setting operation of the extracorporeal circuit does not require disposing or positioning the venous reservoir, the oxygenator, and the pump. This allows a relatively quick preparatory use of the extracorporeal circuit.

According to another aspect, an oxygenator unit comprises a venous reservoir enclosing an interior, wherein the venous reservoir comprises a reservoir side blood inlet communicating with the interior and a reservoir side blood outlet communicating with the interior, with the reservoir side blood inlet being positioned vertically higher than the reservoir side blood outlet, a centrifugal pump comprising a pump side blood inlet by which blood enters the pump and a pump side blood outlet by which blood exits the pump, and an oxygenator comprising a housing, a gas inlet extending from the housing through which gas is introduced into the oxygenator to effect oxygenation of blood introduced into the oxygenator, a gas outlet extending from the housing through which gas exits the oxygenator, an oxygenator side blood inlet extending from the housing through which blood flows into the oxygenator to be oxygenated, and an oxygenator side blood outlet by which oxygenated blood flows out of the oxygenator. The reservoir side blood outlet is in fluid communication with the pump side blood inlet, and the pump side blood outlet is in fluid communication with the oxygenator side blood inlet. The oxygenator unit also comprises a reservoir receiving frame part in which the venous reservoir is separate from and mounted on the reservoir receiving frame part, a pump receiving frame part in which the pump is separate from and mounted on the pump receiving frame part, and an oxygenator receiving frame part, wherein the oxygenator is separate from and mounted on the oxygenator receiving frame part. The reservoir receiving frame part, the pump receiving frame part, and the oxygenator receiving frame part are unitarily formed in one piece from a common material.

### BRIEF DESCRIPTION OF THE DRAWINGS

FIG. 1 is a perspective view of an oxygenator apparatus disclosed here including an oxygenator unit and a holder.

FIG. 2 is another perspective view of the oxygenator apparatus shown in FIG. 1.

FIG. 3 is a perspective view of the unit frame used in the disclosed oxygenator unit.

FIG. 4 is a perspective view of a unit holder used in the oxygenator apparatus.

FIG. 5 is a side view of the oxygenator unit in which the unit frame has been omitted.

FIG. 6 is a front view of a venous reservoir used in the oxygenator unit disclosed here.

FIG. 7 is a cross sectional view of the venous reservoir shown in FIG. 6.

FIG. 8 is a partially broken front view of a centrifugal pump used in the oxygenator unit disclosed here.

FIG. 9 is a plan view of the centrifugal pump shown in FIG. 8.

FIG. 10 is a front view of a heat exchange function-equipped oxygenator used in the oxygenator unit disclosed here.

FIG. 11 is a right-hand side view of the heat exchange function-equipped oxygenator venous reservoir shown in FIG. 10.

FIG. 12 is a cross-sectional view of the heat exchange function-equipped oxygenator shown in FIG. 10.

FIG. 13 is a perspective view of a lock means of the unit holder used in the oxygenator apparatus disclosed here.

FIG. 14 is a front view of an oxygenator apparatus, including an oxygenator unit and a holder, according to another exemplary embodiment.

FIG. 15 is an illustrative view of an oxygenator apparatus including an oxygenator unit and a holder of a still another exemplary embodiment.

FIG. 16 is an illustration of the oxygenator apparatus shown in FIG. 15.

### DETAILED DESCRIPTION

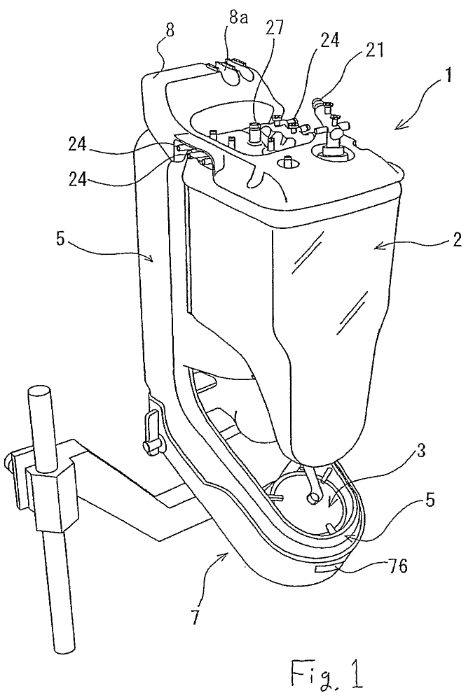

Referring initially to FIGS. 1-5, aspects of the oxygenator apparatus disclosed here are described. The following describes an embodiment of the oxygenator unit disclosed herein and an example of an oxygenator apparatus using the oxygenator unit.

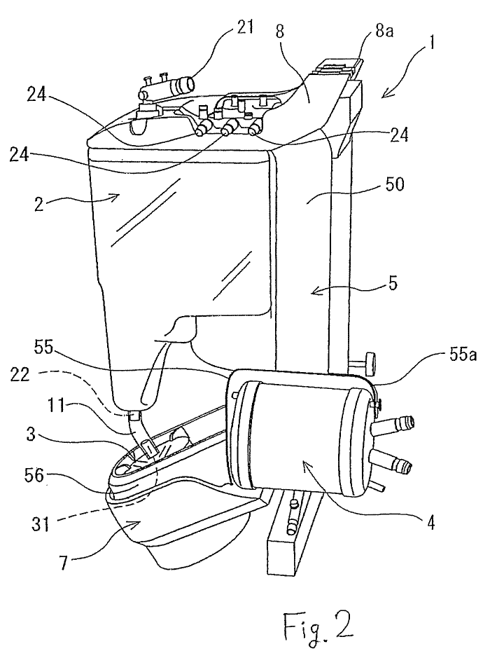

An oxygenator apparatus 1 disclosed here includes, as shown in FIGS. 1 and 2, an oxygenator unit 1, and an oxygenator unit holder 7 capable of having the oxygenator unit 1 mounted on the top surface thereof.

The oxygenator unit 1 includes, as shown in FIGS. 1, 2, and 5, a venous reservoir 2 provided with a reservoir side blood inlet 21 and a reservoir side blood outlet 22, a centrifugal pump 3 provided with a pump side blood inlet 31 and a pump side blood outlet 32, a heat exchange function-equipped oxygenator 4 provided with an oxygenator side blood inlet 41 and an oxygenator side blood outlet 42, a first connection part 11 connecting the reservoir side blood outlet 22 and the pump side blood inlet 31, a second connection part 12 connecting the reservoir side blood outlet 32 and the oxygenator side blood inlet 41, and a unit frame 5. The unit frame 5 is an integrally formed unit. That is, the unit frame 5 is an integral, unitary, one piece unit frame that is entirely formed of the same material and at one time.

The unit frame 5 includes, as shown in FIGS. 1-3, a reservoir receiving frame part (reservoir disposition part) 52, a pump receiving frame part (pump receiving frame part) 53 positioned under the reservoir side blood outlet 22 of the venous reservoir 2 disposed at the reservoir receiving frame part 52, and an oxygenator receiving frame part (oxygenator disposition part) 54 positioned in the vicinity of the pump side blood outlet 32 of the centrifugal pump 3 disposed at the pump receiving part 53.

The venous reservoir 2 is separate from the reservoir disposition part 52 (i.e., the venous reservoir 2 is not integrally formed in one piece with the reservoir disposition part 52) and is disposed or received at the reservoir disposition part 52 of the unit frame and is held by the unit frame 5. The centrifugal pump 3 is separate from the pump disposition part 53 (i.e., the centrifugal pump 3 is not integrally formed in one piece with the pump disposition part 53) and is disposed or received at the pump disposition part 53 of the unit frame and is held by the unit frame 5. In addition, the heat exchange function-equipped oxygenator 4 is separate from the oxygenator receiving part 54 (i.e., the heat exchange function-equipped oxygenator 4 is not integrally formed in one piece with the oxygenator receiving part 54) and is disposed or received at the oxygenator receiving part 54 of the unit frame and is held by the unit frame 5. As a result, these components (the venous reservoir 2, the centrifugal pump 3, and the heat exchange function-equipped oxygenator 4) are integrated together as a single unit, by way of the unit frame 5.

The oxygenator unit holder 7 includes, as shown in FIGS. 1, 2, and 4, a holder main body 71 provided with a unit mounting part 78 configured to receive the oxygenator unit 1 so that the oxygenator unit 1 is mounted on the top surface part of the unit mounting part 78. The oxygenator unit holder 7 also includes a centrifugal pump driving part 75 for rotating an impeller stored in the centrifugal pump 3, and a stand attaching arm member 72 fixed to the holder main body 71.

In the example of the oxygenator unit 1 shown in FIGS. 1 and 2, the top surface of the venous reservoir 2 includes, as described in more detail later, the blood inlet 21 for receiving blood flowing from a venous cannula inserted in the heart, a plurality of cardiotomy blood inlets 24 for receiving the blood from the cardiotomy line for supplying the blood flowing from the operating field, and an air discharge port 27. The oxygenator unit 1 includes a cover member 8 which covers the top surface of the venous reservoir 2 without inhibiting the connection of the tubes to the plurality of blood inlets 21 and 24, and the air discharge port 27.

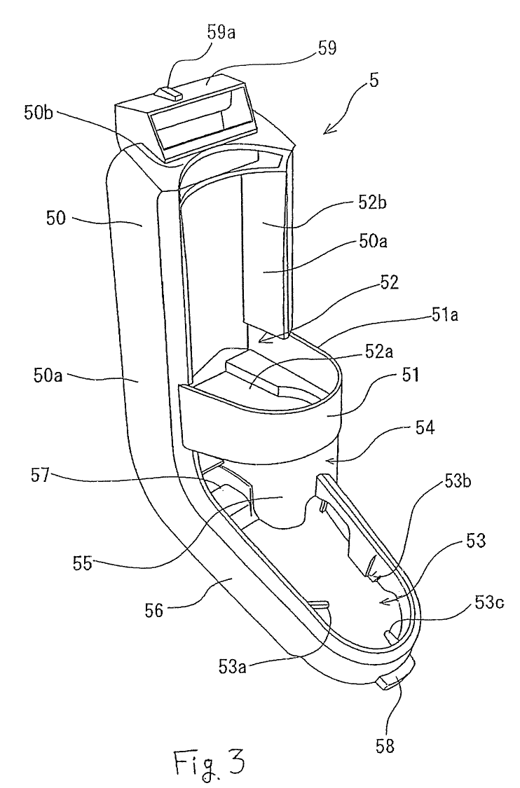

Referring to FIG. 3, the unit frame 5 includes a main frame part 50 extending in the vertical direction, a protruding frame part (laterally protruding frame part) 51 protruding in the horizontal direction from the main frame part 50, a semi tubular part 55 provided at the lower part of the main frame part 50, and situated under the protruding frame part 51, and an extended frame part 56 extending diagonally downward from the lower part of the main frame part 50 so that it extends beyond the protruding frame part 51 (farther outwardly away from the protruding frame part 51).

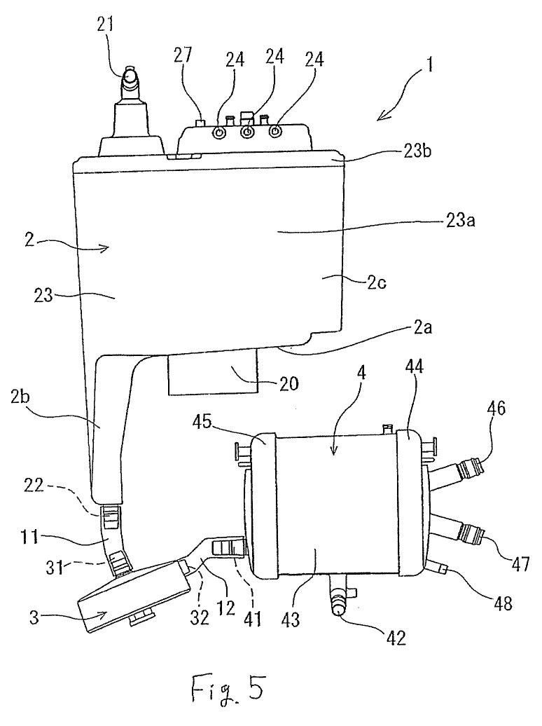

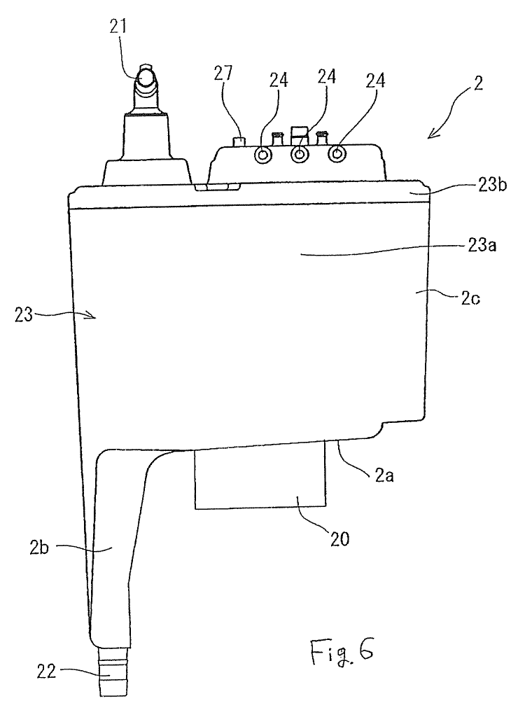

The venous reservoir 2 includes, as shown in FIGS. 5 and 6, a slightly inclined flat bottom surface part 2a, a downwardly protruding part 2b extending further downwardly from the distal end side of the flat bottom surface part 2a, and a rear part 2c extending upwardly from the rear end side of the downwardly protruding part 2b. The lower end of the downwardly protruding part 2b is provided with the reservoir side blood outlet 22. The reservoir 2 includes a bottom member 20 fixed to the flat bottom surface part 2a. The bottom member 20 is formed as a tube-shaped member in which the upper end corresponds to the shape of the slightly inclined flat bottom surface part 2a, and is slightly inclined (slightly tilted with respect to the angle orthogonal to the central axis), and the lower end is roughly horizontal (orthogonal to the central axis). The downwardly protruding part 2b more downwardly protrudes than the lower end of the bottom member 20.

The reservoir disposition part 52 of the unit frame 5 includes, as shown in FIG. 3, a bottom mounting part 52a for mounting the bottom (specifically, the bottom member 20) of the reservoir 2 thereon, and a rear part storage part (recessed part) 52b for storing therein the rear part 2c of the reservoir 2. Specifically, the main frame part 50 of the unit frame 5 includes plate-shaped side parts 50a facing each other and extending in the vertical direction, and a beam part 50b connecting the upper ends of the plate-like parts 50a. The rear part storage part 52b for storing the rear part 2c of the venous reservoir is formed by the two opposing plate-like parts 50a and the beam part 50b. Further, the unit frame 5 includes the protruding frame part 51 protruding in the horizontal direction from a position above the lower end of the main frame part 50 by a prescribed length. The protruding frame part 51 includes a curved wall part 51a including a beginning edge at one plate-like part 50a of the main frame part and terminating at the other plate-like part 50a of the main frame part, and a bottom plate part provided on the lower part side of the curved wall part 51a. Accordingly, the protruding frame part 51 forms a concave part (recessed part) with the rear opened. The concave part forms the bottom mounting part 52a. Therefore, the unit frame 5 is configured to have the reservoir disposition part 52 formed of the main frame part 50 and the protruding frame part 51.

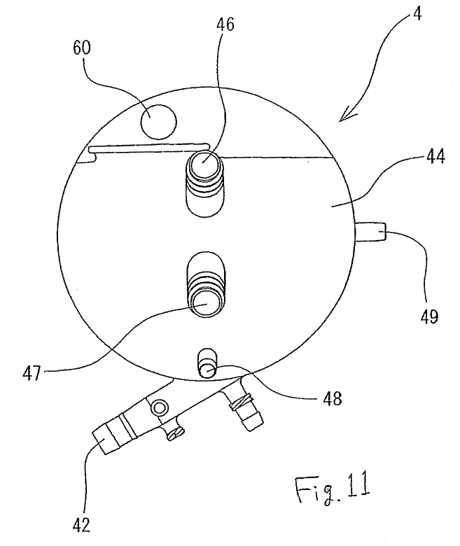

The unit frame 5 includes, as shown in FIGS. 2 and 3, the semi tube-shaped part 55 provided at the lower part of the main frame part 50 and situated under the protruding frame part 51. The oxygenator disposition part 54 is formed by the inside of the semi tube-shaped part 55. The semi tube-shaped part 55 forming the oxygenator disposition part 54 is in a form capable of storing (receiving or holding) the main part of the heat exchange function-equipped oxygenator 4. FIG. 2 shows the oxygenator 4 stored or held in the semi tube-shaped part 55. The semi tube-shaped part 55 includes a holder part 55a for holding a protruding part 60 (shown in FIG. 10) provided at a header 44 of the oxygenator 4. When the oxygenator 4 is stored in the semi tube-shaped part 55, as shown in FIG. 2, various parts of the oxygenator 4 such as the blood outlet port, the heat medium inlet port, and the heat medium outlet port are exposed.

The unit frame 5 includes, as shown in FIGS. 1-3, the extended frame part 56 extending obliquely (or diagonally) downwardly from the lower part of the main frame part 50 in such a manner as to extend beyond the protruding frame part 51. The centrifugal pump disposition part 53 is disposed at the distal end part of the extended frame part 56. The centrifugal pump is stored in the centrifugal pump disposition part 53. Further, the unit frame 5 has pump holding ribs 53a, 53b, 53c protruding inwardly from the inner side surface of the extended frame part 56. Thus, the ribs 53a, 53b, 53c regulate the upward movement of the centrifugal pump 3 and inhibit the pump from being inadvertently removed. Further, the unit frame 5 includes an engagement protruding part 58 at the distal end of the extended frame part 56.

With the venous reservoir 2 disposed at the reservoir receiving part 52, the blood outlet 22 of the reservoir 2 is situated above the pump receiving part 53 of the extended frame part 56. The oxygenator receiving part 54 is situated under the reservoir receiving part 52, and is situated behind the pump receiving part 53.

The oxygenator unit 1 includes the cover member 8 covering the top surface of the venous reservoir 2. The cover member 8 includes an engagement part (not shown) which engages an engagement projection 59a provided at the upper end holding part 59 of the unit frame 5. This engagement inhibits in an auxiliary manner the disengagement of the reservoir from the unit frame 5. Further, on the top surface of the cover member 8, a groove-shaped tube mounting part 8a is provided.

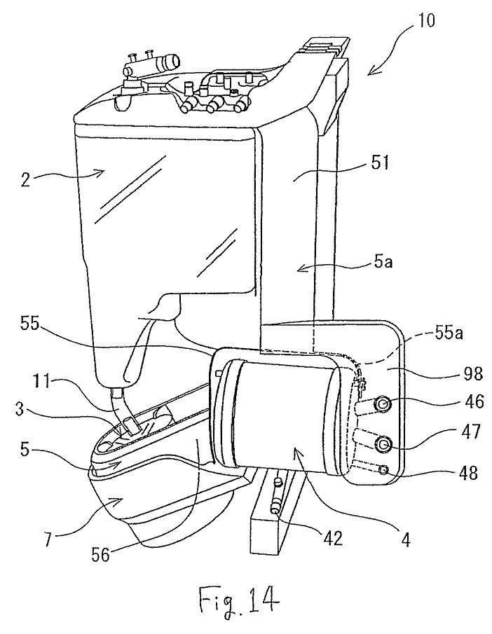

The oxygenator unit may also be an oxygenator unit 10 shown by way of example in FIG. 14. In the oxygenator unit 10, the heat exchange function-equipped oxygenator 4 is configured such that the blood outlet port (oxygenator side blood outlet) 42, the heat exchange medium inlet port 46, the heat exchange medium outlet port 47, and the oxygen-containing gas inlet port 48 are exposed outwardly from a common surface side (front surface side) of the oxygenator unit 10 (the front surface side of FIG. 14). With such a configuration, the tube connection surfaces to the oxygenator unit in use are on the same plane. This facilitates the tube connection operation.

In the oxygenator unit 10, the heat exchange medium inlet port 46, the heat exchange medium outlet port 47, and the oxygen-containing gas inlet port 48 are curved or bent so that their respective openings face the front surface side of the oxygenator unit 10. In addition, the unit frame 5a includes openings for allowing the heat exchange medium inlet port 46, the heat exchange medium outlet port 47, and the oxygen-containing gas inlet port 48 to be placed therein. Specifically, the unit frame 5a includes a plate part 98 having openings for allowing the heat exchange medium inlet port 46, the heat exchange medium outlet port 47, and the oxygen-containing gas inlet port 48 to be placed therein. Further, the blood outlet port 42 provided at the central part of the tube-shaped housing of the heat exchange function-equipped oxygenator 4 is also configured to face outwardly from the front surface side of the oxygenator unit 10.

With such a configuration, the tube connection surfaces to the oxygenator unit in use are on the same plane, and the disposing positions of the port parts are concentrated. This further facilitates the tube connection operation.

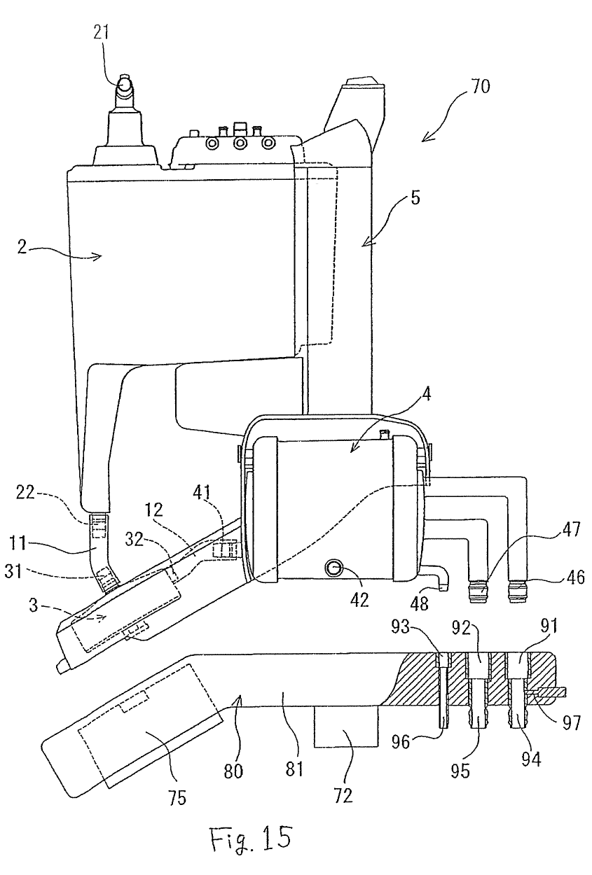

The oxygenator unit may also be an oxygenator unit 70 illustrated by way of example in FIG. 15. In this oxygenator unit 70, the heat exchange medium inlet port 46, the heat exchange medium outlet port 47, and the oxygen-containing gas inlet port 48 of the heat exchange function-equipped oxygenator 4 have openings in a roughly vertical direction and facing downwardly.

The heat exchange medium inlet port 46, the heat exchange medium outlet port 47, and the oxygen-containing gas inlet port 48 in the oxygenator unit are curved or bent such that their respective openings face in the vertical direction of the oxygenator unit 70 and downwardly. In the example shown, the heat exchange medium inlet port 46, the heat exchange medium outlet port 47, and the oxygen-containing gas inlet port 48 are arranged parallel to each other, and in a linear (straight-line) manner.

Also with such a configuration, the tube connection surfaces to the oxygenator unit in use are on the same plane, and the positions of the port parts are also concentrated. This helps facilitate the tube connection operation.

The oxygenator unit is preferably subjected to a sterilization treatment after having been stored in a packed body.

Generally, the oxygenator unit is stored in a packed body which allows a sterile gas to enter therein, and then is subjected to gas sterilization. The packed body may also be further stored in a box. Thus, the packed body may be subjected to gas sterilization while being stored in the box.

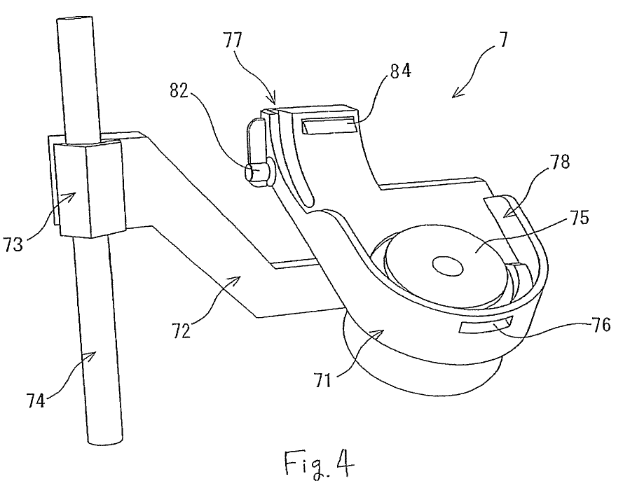

The oxygenator unit holder 7 includes, as shown in FIG. 4, the holder main body 71, the stand attaching arm member 72 fixed to the holder main body 71, and a stand fixing means 73 attached to the arm member 72. Thus, the oxygenator unit holder 7 is fixed to the stand 74 by the stand fixing means. In the illustrated embodiment, the stand fixing means is a bracket.

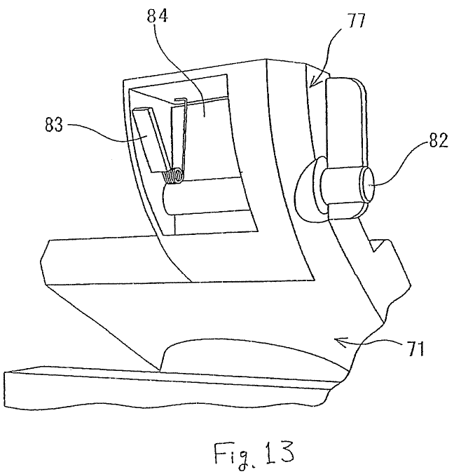

As shown in FIG. 4, the holder main body 71 includes the unit mounting part 78 configured to allow the oxygenator unit 1 to be mounted on the top surface part thereof. The unit mounting part 78 has a shape corresponding to the shape of the bottom surface of the unit frame 5 of the oxygenator unit 1, and is configured as a concave part capable of storing or hold (receiving) the bottom surface part of the extended frame part 56 of the unit frame 5. Specifically, the unit mounting part 78 includes a wall part for storing or receiving the distal end side part of the extended frame part 56. The holder main body 71 includes an engagement part (specifically, a notch part) 76 which engages the engagement protruding part 58 formed at the distal end of the extended frame part 56 of the unit frame 5 of the unit 1. Further, the holder main body 71 includes a lock means 77 for releasably engaging the rear part of the unit frame 5 of the unit 1. The lock means 77 includes, as shown in FIGS. 4 and 13, an engagement nail part 84 projecting from the rear part surface of the holder main body 71 through operation of an operation part 82, and an urging member 83 for urging the engagement nail part 84 in the direction of projection. The engagement nail part 84 is provided in an upstanding wall portion of the holder main body 71. The engagement nail part 84 is configured to project in a manner causing the engagement nail part 84 to engage the rear part edge part 57 of the unit frame 5 shown in FIG. 3. The nail part 84 is urged to the position shown in FIG. 3 (forward position), by the urging member 83. Therefore, the oxygenator unit holder 7 of this example is configured to hold the oxygenator unit 1 in a fixed or unreleasable state at the front side part and the rear side part (i.e., the oxygenator unit 1 is sandwiched from opposite directions by virtue of the engagement nail part 84 engaging the rear part edge part 57 of the unit frame, and the engagement part 76 engaging the engagement protruding part 58). Rotating the operation part 82 in the counter-clockwise direction (e.g., about 90 degrees) with reference to FIG. 3 moves the nail part 84 away from the forward position shown in FIG. 3 against the urging force of the urging member 83 to release the oxygenator unit 1.

Further, the oxygenator unit holder 7 includes, as shown in FIG. 4, a centrifugal pump driving part 75 for rotating the impeller (described later) positioned in the centrifugal pump 3. The impeller of the centrifugal pump has a magnetic member. The centrifugal pump driving part 75 mounted in the holder main body 71 includes a permanent magnet for attracting the magnetic member of the impeller. The oxygenator unit is held in, or is assisted in being held in, the holder by the attraction force attracting the impeller of the centrifugal pump by the permanent magnet of the centrifugal pump driving part.

Further, the holder may also be one having a port mounting part as shown in FIG. 15. The holder 80 in the oxygenator apparatus of this example is for supporting the oxygenator unit 70 shown in FIG. 15.

Also in the holder 80 of this example, the holder main body 81 includes a unit mounting part capable of mounting the oxygenator unit 70 on the top surface part thereof, and a centrifugal pump driving part 75 for rotating an impeller housed in the centrifugal pump 3.

Further, the holder 80 includes a heat exchange medium inlet port mounting part 91 adapted to mount the heat exchange medium inlet port 46 on the top surface of the holder, a heat exchange medium outlet port mounting part 92 adapted to mount the heat exchange medium outlet port 47, and a gas inlet port mounting part 93 adapted to mount the oxygen-containing gas inlet port 48. Specifically, the holder main body 81 includes three concave parts (recessed portions) extending from the top surface in the vertical direction (vertical and downward direction). The concave parts each include a flexible tubular body inserted and fixed thereto. Then, the first concave part and the flexible tube body inserted and fixed in the inside thereof form the heat exchange medium inlet port mounting part 91. Similarly, the second concave part and the flexible tube body inserted and fixed in the inside thereof form the heat exchange medium outlet port mounting part 92. Further, the third concave part and the flexible tube body inserted and fixed in the inside thereof form the gas inlet port mounting part 93.

Further, the holder main body 81 includes a heat exchange medium inlet 94 communicating with the heat exchange medium inlet port mounting part 91. In this example, the heat exchange medium inlet 94 is a protruding part protruding downwardly from the bottom surface of the holder main body 81, and hence it can be connected with the heat medium supply unit with relative ease. Similarly, the holder main body 81 includes a heat exchange medium outlet 95 communicating with the heat exchange medium outlet port mounting part 92. In this example, the heat exchange medium outlet 95 is a protruding part protruding downwardly from the bottom surface of the holder main body 81, and hence it can be connected with the heat medium supply unit with relative ease. Then, the holder main body 81 includes a gas inlet 96 communicating with the gas inlet port mounting part 93. In this example, the gas inlet 96 is a protruding part protruding downwardly from the bottom surface of the holder main body 81, and hence it can be connected with the oxygen-containing gas supply unit with relative ease.

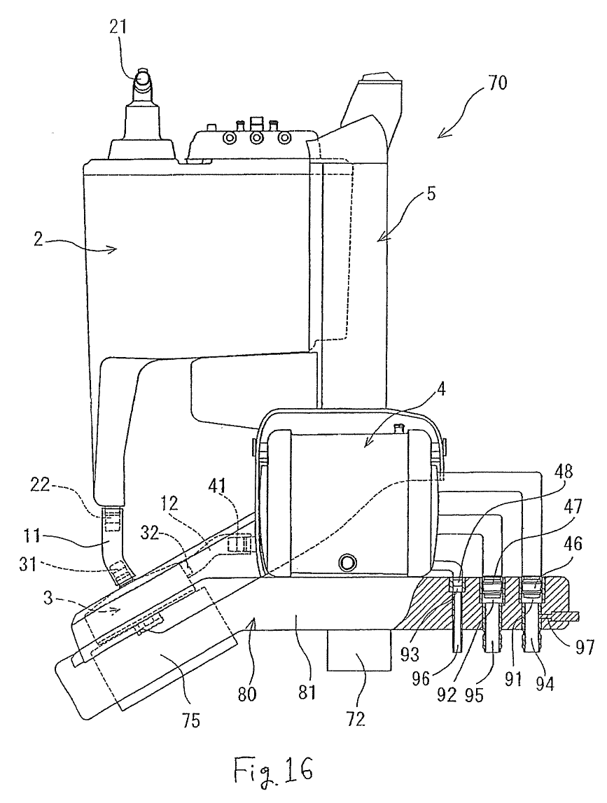

With the oxygenator apparatus of this example, as shown in FIG. 16, by mounting the oxygenator unit 70 onto the top surface part of the holder 80, the mounting of the heat exchange medium inlet port 46 to the heat exchange medium inlet port mounting part 91, the mounting of the heat exchange medium outlet 47 to the heat exchange medium outlet port mounting part 92, and the mounting of the oxygen-containing gas inlet port 48 to the gas inlet port mounting part 93 are also simultaneously carried out. This eliminates the necessity of performing individual connection operations to the heat exchange medium inlet port, the heat exchange medium outlet port, and the oxygen-containing gas inlet port of the oxygenator unit 70. Further, it is possible to previously connect the heat medium supply unit to the heat exchange medium inlet 94 and the heat exchange medium outlet 95 of the holder 80, and the oxygen-containing gas supply unit to the gas inlet port 96 thereof. Thus, it is possible to carry out setting of the oxygenator apparatus relatively quickly.

The holder 80 of this example includes a temperature sensor 97 attached to the duct between the heat exchange medium inlet port mounting part 91 and the heat exchange medium inlet 94. The temperature sensor may be attached to the duct between the heat exchange medium outlet port mounting part 92 and the heat exchange medium outlet 95, or may be attached to the ducts of both. Fixing the temperature sensor to the holder 80 eliminates the necessity of performing an attachment operation of the temperature sensor to the oxygenator unit. Further, setting of the oxygenator apparatus can be carried out more quickly. Known temperature sensors can be used here. A preferred temperature sensor is one using a thermocouple.

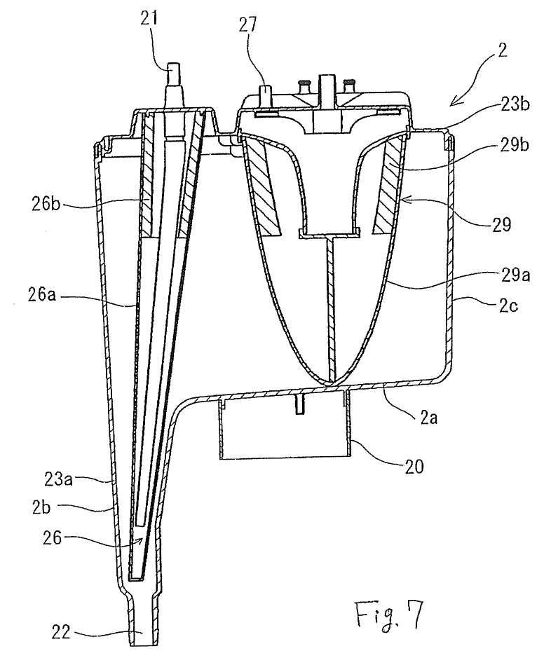

Set forth below is a description of the venous reservoir 2 used in the oxygenator unit 1 disclosed here. As shown in FIGS. 6 and 7, the venous reservoir 2 comprises a housing 23 including a reservoir housing main body 23a formed of a hard resin, a cover body 23b, and the bottom member 20 attached to the bottom surface of the housing 23.

The cover body 23b is fitted to the upper end of the housing main body 23a to cover the upper opening of the reservoir housing main body 23a. The cover body 23b includes the blood inlet 21 to be connected with a venous line for supplying blood from the venous cannulae inserted into the heart ascending and descending veins of the patient, the blood inlet 24 to be connected with the cardiotomy line for supplying blood from the operating field, and the air discharge port 27 also usable as a rapid priming port.

A blood filter (venous blood filter) 26 is positioned in the housing 23. The blood filter serves as a defoaming member and anti-foaming member for filtrating the blood flowing from the blood inlet port (reservoir side blood inlet) 21. Also located within the housing 23 is a cardiotomy blood filter 29 for filtrating the blood flowing from the blood inlet port 24. The lower end of the blood inlet port 21 extends in the blood reservoir, and is situated in the blood filter 26. All the blood flowing from the blood inlet port 21 passes through the blood filter 26, and then flows into the blood reservoir. Specifically, the blood filter 26 is fixed so that the protruding part of the blood inlet port 21 which protrudes into the blood reservoir is encapsulated in the inner side of the cover body 23b.

The reservoir housing main body 23a includes the protruding part 2b which protrudes downwardly. The lower end of the blood filter 26 extends to the vicinity of the lower end of the protruding part 2b. The blood filter 26 includes a filter member 26a and a defoaming material 26b attached to the entire inner circumference of the upper part of the filter member 26a. Similarly, the cardiotomy blood filter 29 includes a filter member 29a and a defoaming material 29b attached to the entire inner circumference of the upper part of the filter member 29a.

Defoaming materials 26b, 29b, or anti-foaming materials, which can be used preferably include various porous bodies such as foamed products including foamed polyurethane, foamed polyethylene, foamed polypropylene, foamed polystyrene, and the like, and mesh, web, nonwoven fabric, or porous ceramics, and sintered bodies of resins. Particularly preferable are materials having a relatively low air pass resistance (pressure loss). When a foamed product such as foamed polyurethane or other porous materials are used as the material having a low air pass resistance, the pore diameter thereof is preferably 20 μm to 10 mm, and in particular, about 50 μm to 5 mm.

Further, such defoaming materials 26b, 29b preferably carry a defoaming agent which function to break bubbles upon contact therewith. As the defoaming agent, silicone (of a silica-mixed compound type, an oil type, or the like) is preferable. Examples of methods for causing the defoaming members 26b, 29b to carry or be provided with a defoaming agent include a method in which a defoaming member is impregnated in a liquid containing a defoaming agent, a method in which a liquid containing a defoaming agent is applied or sprayed, and dried (for example, at 30° C. for 180 minutes), and other methods.

The filter members 26a, 29a are adapted to remove foreign matters or bubbles in blood. As the material for the filter members 26a, 29a, a porous material having sufficient blood permeability is preferable.

As depicted in FIG. 7, the inside of the venous reservoir 2 forms a blood reservoir part for temporarily reserving blood. The volume of the blood reservoir part is not particularly limited. However, it is generally about 3000 to 5000 ml for an adult, and 1000 to 2500 ml for an infant. Whereas, the reservoir housing main body 23a is preferably substantially transparent or translucent so as to allow easy observation of the amount of the reserved blood and the state of the blood reserved in the inside thereof.

The centrifugal pump 3 is disposed under the venous reservoir 2 as shown in FIG. 5. The blood inlet 31 of the centrifugal pump 3 and the blood outlet 22 of the venous reservoir 2 are connected by the first connection part 11, specifically, by the first connection tube. The tube 11 is preferably a flexible resin tube.

Set forth below is a description of the use of the centrifugal pump 3 in the oxygenator unit 1 disclosed here.

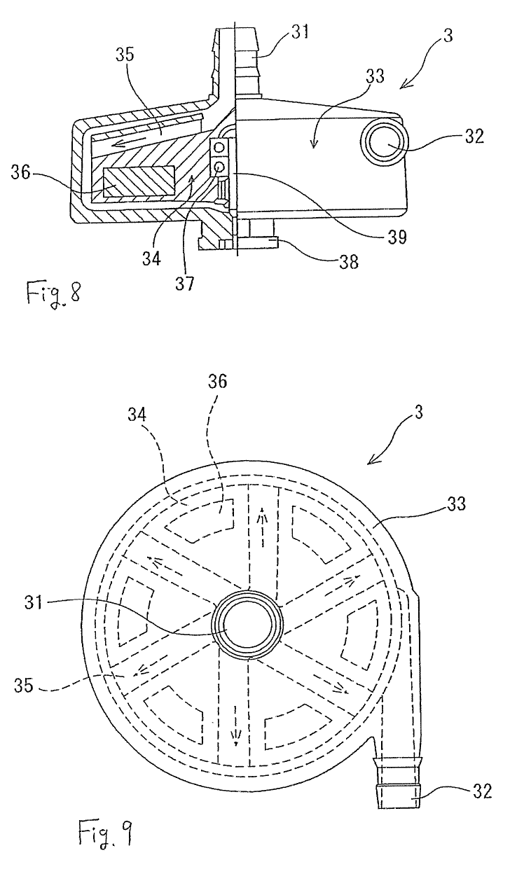

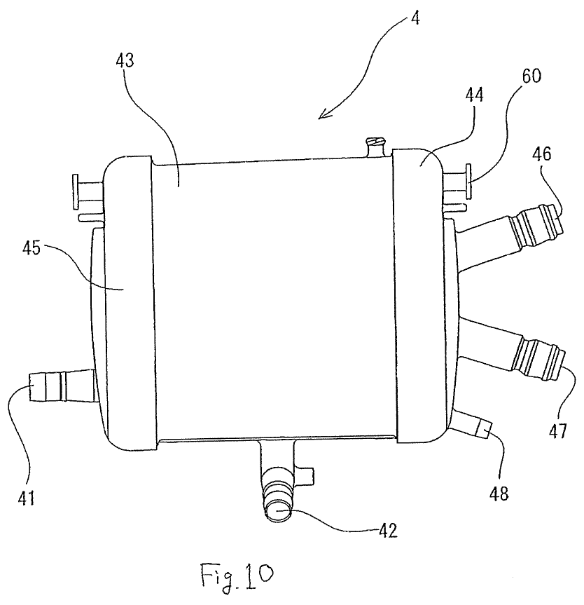

Referring to FIGS. 8 and 9, the centrifugal pump 3 includes a housing 33 and an impeller 34 rotatably housed in the housing 33. The housing 33 includes a blood inlet port 31 protruding upwardly from the center region (e.g., the center) of the housing, a blood outlet port 32 extending from the side in the tangential direction, and a protruding part 38 for engagement with the pump driving part. The impeller 34 is rotatably supported on a shaft 39 by a ball bearing 37. In addition, the impeller 34 rotates with respect to the shaft 39 in a liquid tight manner by the sealing member. The top surface of the impeller 34 is in the shape of a cone. A plurality of blood introduction paths 35 are formed in the impeller 34 and extend from roughly the center of the impeller 34 in the outer circumferential direction. The blood flowing from the blood inlet port is dispersed at the apex portion of the impeller 34, and then is applied with a centrifugal force by rotation of the impeller 34. Thus, the blood flows in the blood introduction paths 35, passes between the outer surface of the impeller 34 and the inner surface of the housing 33, and flows out of the blood outlet port 32. A plurality of magnetic members (specifically permanent magnets) 36 are positioned inside the impeller 34. The magnetic members 36 transfer the rotation force to the impeller 34 from the outside. In the centrifugal pump 3, an impeller 34 that is rotatable at speeds above 0 rpm up to 3000 rpm is used.

The centrifugal pump driving part 75 mounted in the oxygenator unit holder 7 can be of a known type of driving part. The driving part 75 generally includes a motor, a rotational member (e.g., a rotational plate) fixed to the rotation shaft of the motor, and a permanent magnet attached to the rotational plate. The permanent magnet is provided at the position corresponding to the magnetic member 36 provided in the impeller 34. For this reason, the rotational plate of the driving part 75 is connected by the magnetic attraction force through the housing to the impeller of the centrifugal pump. Then, rotation of the motor causes the rotational plate to rotate. The impeller 34 also rotates following the rotation of the rotational plate. The motor can be, for example, an AC motor, a DC motor, and the like. However, a variable speed motor is preferable. Further, a motor which is easily controlled in flow rate is preferable. For example, a stepping motor which is an AC motor is preferable.

Referring to FIGS. 10-12, set forth below is a description of the heat exchange function-equipped oxygenator used in the oxygenator unit 1 disclosed here.

The heat exchange function-equipped oxygenator 4 is disposed at a position in the vicinity of the pump side blood outlet of the centrifugal pump 3, specifically, behind the centrifugal pump 3. Stated differently, the heat exchange function-equipped oxygenator 4 is positioned downstream of the centrifugal pump 3 The blood outlet 32 of the centrifugal pump 3 is connected with the blood inlet 41 of the heat exchange function-equipped oxygenator 4 by the second connection part 12, specifically the second connection tube. The tube 12 is preferably a flexible resin tube.

The heat exchange function-equipped oxygenator 4 includes, as shown in FIGS. 10-12: a tube-shaped heat exchange unit part 17; a tube-shaped hollow fiber membrane bundle 15 including a large number of gas exchange hollow fiber membranes in which is housed the tube-shaped heat exchange unit part 17; a housing 43 in which is housed the tube-shaped hollow fiber membrane bundle 15 and the tube-shaped heat exchange unit part 17; two partitions 85, 86 which fix opposite ends of the tube-shaped hollow fiber membrane bundle 15 to the housing 43 and the tube-shaped heat exchange unit part 17 so that the opposite ends of the hollow fiber membrane are open; a blood chamber 62 formed between the inner surface of the housing 43, the outer surface of the hollow fiber membrane, the outer side surface of the tube-shaped heat exchange unit part 17, and the two partitions 85, 86; a blood inlet 41 and a blood outlet 42 in the housing 43 communicating with the blood chamber 62; a gas inlet 48 and a gas outlet 49 communicating with the inside of the hollow fiber membrane (the interior of the gas exchange hollow fiber membranes); and a heat medium inlet 46 and a heat medium outlet 47 communicating with the inside of the tube-shaped heat exchange unit part 17.

In the heat exchange function-equipped oxygenator 4, the housing 43 includes a tube-shaped (tubular) housing main body, a first header 44 including the gas inlet 48 and the heat medium inlet 46, and the heat medium outlet 47, and a second header 45 having the gas outlet 49. The blood inlet port 42 is provided at a position which is the lower end (lower half) of the central part (central part relative to the length of the housing) of the side surface of the tube-shaped housing main body.

The tube-shaped hollow fiber membrane bundle 15 is wound around the outer surface of the core 67 including a large number of openings 68. Further, the tube-shaped heat exchange unit part 17 is positioned in the inside of the core 67. A blood circulation part 61 (which is also a part of the blood chamber) is located between the inner surface of the core 67 and the outer surface of the tube-shaped heat exchange unit part 17. The blood circulation part 61 communicates with the blood inlet 41 and the blood outlet 42. Heat medium circulation chambers 65, 66 are located in the inside of the tube-shaped heat exchange unit part 17. The heat medium circulation chambers 65, 66 communicate with the heat medium inlet 46 and the heat medium outlet 47.

In the heat exchange function-equipped oxygenator 4, blood flowing from the blood inlet 41 into the oxygenator 4 is subjected to heat exchange while flowing between the inner surface of the core 67 and the outer surface of the tube-shaped heat exchange unit part 17. The blood subjected to heat exchange flows from the openings 68 of the core 67 into the tube-shaped hollow fiber membrane bundle 15 (into the blood chamber). As the blood contacts the hollow fiber membrane, gas exchange in the blood is carried out (i.e., oxygen is added and carbon dioxide is removed). Then, the blood which has passed through the tube-shaped hollow fiber membrane bundle 15 flows into the annular space (which is a part of the blood chamber) formed between the tube-shaped hollow fiber membrane bundle 15 and the inner surface of the tube-shaped housing main body, and flows out of the blood outlet 42.

As the hollow fiber membrane for gas exchange, a porous film is preferably used.

The tube-shaped heat exchange unit part 17 is preferably a so-called bellows type heat exchange part. The bellows type tube-shaped member, which is at least the outer side surface forming member of the tube-shaped heat exchange unit part 17, is formed in the shape of so-called fine bellows with a metal such as stainless steel or aluminum or a resin material such as polyethylene or polycarbonate. A metal such as stainless steel or aluminum is preferred from the viewpoints of the strength and the heat exchange efficiency. In this example, the tube-shaped heat exchange unit part 1 is one including a bellows tube in the shape of a wave including a large number of repeating projections and depressions roughly orthogonal to the axial direction (central axis) of the tube-shaped heat exchange unit part.

The heat exchange unit part 17 includes the bellows type tube-shaped member forming the outer side surface, and an inner side tube-shaped member 63 stored in the inside of the bellows type tube-shaped member. A heat medium chamber is formed between the inner side tube-shaped member and the bellows type tube-shaped member. Further, the inner side tube-shaped member 63 has a bottom surface closed, and has two openings 64a, 64b on the side surface thereof, and the heat medium inlet 46 and the heat medium outlet 47. Further, the inside of the inner side tube-shaped member 63 is divided into two internal chambers 65 and 66. The heat medium inlet 46 communicates with the internal chamber 65, and the heat medium outlet 47 communicates with the second internal chamber 66. With the heat exchange unit part 17, the heat exchange medium flowing from the heat medium inlet 46 flows into the first internal chamber 65, and passes through the opening 64a, and flows between the bellows type tube-shaped member and the inner side tube-shaped member 63. Then, the heat medium flows from the opening 64b into the second internal chamber 66, and flows out of the heat medium outlet 48.

The inner side tube-shaped member is formed of a synthetic resin or a metal such as stainless steel or aluminum. Whereas, the tube-shaped heat exchange unit part 17 is preferably in the shape of a cylinder.

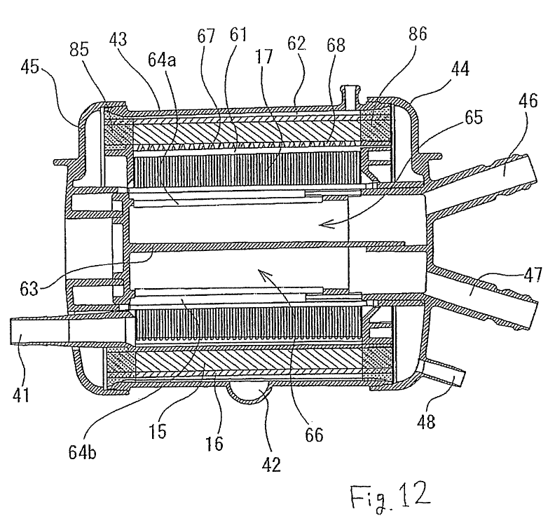

Further, the heat exchange function-equipped oxygenator 4 of this example includes an arterial filter function. Specifically, as shown in FIG. 12, a filter member 16 is positioned on the outer circumferential part of the tube-shaped hollow fiber membrane bundle 15 (i.e., the filter member 16 encircles the outer circumferential surface of the tube-shaped hollow fiber membrane bundle 15). The filter member 16 traps bubbles in the blood flowing into the oxygenator 4. The filter member 16 has an overall cylindrical shape as with the tube-shaped hollow fiber membrane bundle 15. The filter member 16 is positioned in such a manner that the inner circumferential part (inner circumferential surface) is in contact with the outer circumferential part (outer circumferential surface) of the tube-shaped hollow fiber membrane bundle 15. Further, the filter member 16 covers the entire outer circumferential part of the tube-shaped hollow fiber membrane bundle 15 (inclusive of substantially the entire outer circumferential part of the tube-shaped hollow fiber membrane bundle 15).

As shown in FIG. 12, the blood chamber 62 (gap) is provided between the filter member 16 and the housing 43. By virtue of the blood chamber or gap 62, the filter member 16 is not in contact with the inner surface of the housing 43. The filter member 16 preferably allows blood to pass therethrough, and has a hydrophilicity (possesses hydrophilic properties). The material forming the filter member 16 is not limited to a particular material. An example of a preferable material is a material in mesh form (screen filter). This type of material can trap bubbles with more reliability, while allowing blood to pass therethrough with ease. The filter member 16 with such a configuration can trap bubbles in the blood flowing from the blood outlet 41 with reliability. Further, the bubbles trapped by the filter member 16 enter (flow into) a large number of holes formed in the hollow fiber membrane, and are thus discharged from the gas outlet port through the bore of the hollow fiber membrane.

It is preferable that the blood contact surfaces of the centrifugal pump and the oxygenator are antithrombogenic surfaces. The antithrombogenic surface formation can be accomplished by the use of a known method.

The principles, embodiments and modes of operation of the oxygenator unit and oxygenator apparatus have been described in the foregoing specification, but the invention which is intended to be protected is not to be construed as limited to the particular embodiments disclosed. The embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. An oxygenator unit comprising:
a venous reservoir enclosing an interior, the venous reservoir comprising a reservoir side blood inlet communicating with the interior and a reservoir side blood outlet communicating with the interior, the reservoir side blood inlet being positioned vertically higher than the reservoir side blood outlet;
a centrifugal pump comprising a pump side blood inlet by which blood enters the pump and a pump side blood outlet by which blood exits the pump;
an oxygenator comprising a housing, a gas inlet extending from the housing through which gas is introduced into the oxygenator to effect oxygenation of blood introduced into the oxygenator, a gas outlet extending from the housing through which gas exits the oxygenator, an oxygenator side blood inlet extending from the housing through which blood flows into the oxygenator to be oxygenated, an oxygenator side blood outlet by which oxygenated blood flows out of the oxygenator;
the reservoir side blood outlet being in fluid communication with the pump side blood inlet;
the pump side blood outlet being in fluid communication with the oxygenator side blood inlet;
a reservoir receiving frame part, the venous reservoir being separate from and mounted on the reservoir receiving frame part;
a pump receiving frame part, the pump being separate from and mounted on the pump receiving frame part;
an oxygenator receiving frame part, the oxygenator being separate from and mounted on the oxygenator receiving frame part; and
the reservoir receiving frame part, the pump receiving frame part, and the oxygenator receiving frame part being unitarily formed in one piece from a common material.

2. The oxygenator unit according to claim 1, wherein the pump and the pump receiving frame part are located under the reservoir side blood outlet of the venous reservoir mounted at the reservoir receiving frame part.

3. The oxygenator unit according to claim 1, wherein the reservoir receiving frame part is a horizontally extending reservoir receiving frame part that protrudes horizontally from a main frame part, the main frame part comprising a recessed part that receives a rear portion of the venous reservoir.

4. The oxygenator unit according to claim 3, wherein the oxygenator receiving frame part is a semi tube-shaped oxygenator receiving frame part positioned under the protruding frame part, and further comprising an extended frame part extending obliquely downwardly from a lower part of the main frame part so that the extended frame part extends outwardly beyond the protruding frame part, the pump receiving frame part being located at a distal end of the extended frame part.

5. The oxygenator unit according to claim 4, further comprising a distally extending protruding part positioned at the distal end of the extended frame part for engaging a groove provided in a holder on which the oxygenator unit is to be mounted.

6. The oxygenator unit according to claim 1, wherein the oxygenator is a heat exchange function-equipped oxygenator from which extends a heat exchange medium inlet port to introduce a heat exchange medium into the heat exchange function-equipped oxygenator and a heat exchange medium outlet port through which the heat exchange medium flows away from the heat exchange function-equipped oxygenator.

7. An oxygenator unit comprising:
a venous reservoir enclosing a reservoir interior, the venous reservoir comprising a reservoir side blood inlet communicating with the reservoir interior and a reservoir side blood outlet communicating with the reservoir interior;
a centrifugal pump comprising a pump side blood inlet by which blood enters the pump and a pump side blood outlet by which blood exits the pump;
a heat exchange function-equipped oxygenator comprising an oxygenator side blood inlet by which blood is introduced into the heat exchange function-equipped oxygenator to be oxygenated and subjected to heat-exchange, and an oxygenator side blood outlet by which oxygenated and heat-exchanged blood flows out of the heat exchange function-equipped oxygenator;
a first connection part connecting the reservoir side blood outlet and the pump side blood inlet;
a second connection part for connecting the pump side blood outlet and oxygenator side blood inlet;
an integral, unitary, one piece unit frame formed entirely at one time;
the unit frame comprising a reservoir disposition part at which is disposed the venous reservoir, a pump disposition part at which is disposed the pump, and an oxygenator disposition part at which is disposed the oxygenator, the pump disposition part being positioned under the reservoir side blood outlet of the venous reservoir disposed at the reservoir disposition part, and the oxygenator disposition part being positioned in a vicinity of the pump side blood outlet of the centrifugal pump disposed at the pump disposition part; and
the venous reservoir being held by the unit frame, the centrifugal pump being held by the unit frame, and the heat exchange function-equipped oxygenator being held by the unit frame to thereby result in a unit.

8. The oxygenator unit according to claim 7, wherein the unit frame comprises a vertically extending main frame part, a protruding frame part extending horizontally from the main frame part, a semi tube-shaped part positioned at a lower part of the main frame part under the protruding frame part, and an extended frame part extending obliquely downwardly from the lower part of the main frame part so that the extended frame part extends outwardly beyond the protruding frame part, a top surface of the protruding frame part providing the reservoir disposition part at which is positioned the venous reservoir, the semi tube-shaped part forming the oxygenator disposition part at which is received the heat exchange function-equipped oxygenator, and a distal end portion of the extended frame part forming the pump disposition part at which is received the pump.

9. The oxygenator unit according to claim 8, wherein the venous reservoir comprises a bottom part and a downwardly protruding part extending downwardly from the bottom part so that the downwardly protruding part extends further downwardly than the bottom part, the reservoir side blood outlet being positioned at a lower end of the downwardly protruding part, the bottom part of the venous reservoir being positioned on the top surface of the reservoir disposition part, and the downwardly protruding part including the reservoir side blood outlet of the venous reservoir being positioned above the pump disposition part formed by the extended frame part.

10. The oxygenator unit according to claim 7, wherein the oxygenator receiving part is located under the reservoir disposition part.

11. The oxygenator unit according to claim 7, wherein the venous reservoir comprises a top surface at which is positioned the reservoir side blood inlet in which blood from a venous cannula to be inserted in a heart is adapted to flow, a plurality of cardiotomy blood inlets in which blood from a cardiotomy line for supplying blood from an operating field is adapted to flow, and an air discharge port, and further comprising a cover member covering the top surface of the venous reservoir without interfering with connection of tubes to the cardiotomy and reservoir side blood inlets, and the air discharge port.

12. The oxygenator unit according to claim 7, wherein the heat exchange function-equipped oxygenator comprises an interiorly located filter which is adapted to trap bubbles.

13. The oxygenator unit according to claim 7, wherein the heat exchange function-equipped oxygenator comprises a heat exchange medium inlet port, a heat exchange medium outlet port, and an oxygen-containing gas inlet port; and the oxygenator side blood outlet port, the heat exchange medium inlet port, the heat exchange medium outlet port, and the oxygen-containing gas inlet port are all exposed outwardly from a common front side of the exchange function-equipped oxygenator.

14. The oxygenator unit according to claim 7, wherein the heat exchange function-equipped oxygenator comprises a heat exchange medium inlet port, a heat exchange medium outlet port, and an oxygen-containing gas inlet port; and the heat exchange medium inlet port, the heat exchange medium outlet port, and the oxygen-containing gas inlet port all protrude from a common surface of the unit frame.

15. The oxygenator unit according to claim 7, wherein the heat exchange function-equipped oxygenator comprises a heat exchange medium inlet port, a heat exchange medium outlet port, and an oxygen-containing gas inlet port; and the oxygenator side blood outlet port, the heat exchange medium inlet port, the heat exchange medium outlet port, and the oxygen-containing gas inlet port each have an opening facing vertically downward.

16. A oxygenator apparatus, comprising:
the oxygenator unit according to claim 7; and
an oxygenator unit holder comprised of a holder main body including a unit mounting part at which is mounted the oxygenator unit, a centrifugal pump driving part for rotating an impeller in the centrifugal pump, and a stand attaching member fixed to the holder main body, wherein the impeller has a magnetic member, the centrifugal pump driving part includes a permanent magnet for attracting the magnetic member of the impeller, and the oxygenator unit is mounted to the oxygenator unit holder by the attraction force for attracting the impeller of the centrifugal pump by the permanent magnet of the centrifugal pump driving part.

17. The oxygenator apparatus according to claim 16, wherein the holder includes a lock mechanism releasably engaging the unit frame.

18. The oxygenator apparatus according to claim 16, wherein the oxygenator unit holder comprises a heat exchange medium inlet port mounting part to which is mounted the heat exchange inlet port of the heat exchange function-equipped oxygenator, a heat exchange medium outlet port mounting part to which is mounted to the heat exchange medium outlet port, and a gas inlet port mounting part to which is mounted the oxygen-containing gas inlet port, and further comprises a heat exchange medium inlet communicating with the heat exchange medium inlet port mounting part, a heat exchange medium outlet communicating with the heat exchange medium outlet port mounting part, and a gas inlet communicating with the oxygen-containing gas inlet port mounting part.

19. The oxygenator apparatus according to claim 18, wherein the holder comprises a temperature sensor attached to a duct between the heat medium inlet port mounting part and the heat exchange medium inlet, or attached to a duct between the heat exchange medium outlet port mounting part and the heat exchange medium outlet.
